(19) United States
(12) Patent Application Publication
(10) Pub. No.: US 2011/0311587 A1
(43) Pub. Date: Dec. 22, 2011

(54) Fusogenic virus-like particles and uses thereof

(76) Inventor: Pramila Walpita, Galveston, TX (US)

(21) Appl. No.: 13/134,266

(22) Filed: Jun. 3, 2011

Related U.S. Application Data
(60) Provisional application No. 61/396,915, filed on Jun. 4, 2010.

(51) Int. Cl. A61K 39/155; A61P 37/04; A61P 31/14; C12N 15/63

(52) U.S. Cl. 424/211.1; 435/320.1

(57) ABSTRACT

Provided herein are novel Paramyxovirus virus-like particles, wherein said virus-like particles are composed of surface glycoprotein G; surface glycoprotein F; and matrix protein M. Further provided is a vaccine comprising the virus-like particles described herein and a pharmaceutically acceptable carrier. Also provided is a method of vaccinating a subject against paramyxovirus infection comprising administering the vaccine described herein.

### CROSS-REFERENCE TO RELATED APPLICATIONS

This nonprovisional application claims benefit of priority under 35 U.S.C. §119(e) of provisional application U.S. Ser. No. 61/396,915, filed Jun. 4, 2010 now abandoned, the entirety of which is hereby incorporated by reference.

### FEDERAL FUNDING

The invention was supported, in whole or in part, by Grant No. U54 AI057156-07 from the National Institutes of Health. Consequently, the Government has certain rights in the invention.

### BACKGROUND OF THE INVENTION

1. Field of the Invention

The present invention relates to the field of paramyxovirus biology. More specifically, the present invention relates to nipah virus-like particles and uses thereof.

2. Description of the Related Art

Since it was first recognized in 1998, Nipah virus (NiV) has caused several outbreaks in humans of encephalitic disease associated with high lethality. In the first outbreak, which was in Malaysia and Singapore, 265 humans became sick and some ˜40% of them died. Epidemiological links pointed to human contact with sick pigs in commercial piggeries, and the outbreak was brought under control through culling of approximately ˜1.1 million pigs [1,2,3,4]. Since then, the virus has re-emerged in Bangladesh and India, starting in 2001, and has caused several smaller but even deadlier outbreaks with fatality rates between 60 and 90% [5,6,7,8]. Unlike the Malaysian outbreak, the route of transmission in these outbreaks was considered to be bat-to-human via food contaminated with bat saliva [9]. In some cases, nosocomial transmissibility and person-to-person spread was also noted [5,10,11,12]. An additional concern is that Nipah virus is a potential agro-terror agent since the rate of transmission of this virus in the pig population is close to 100% [13]. Effective vaccine and therapies are needed to combat the threats posed by Nipah virus.

Nipah virus is a member of the genus Henipavirus in the subfamily Paramyxovirinae, family Paramyxoviridae. It has several distinctive genetic and biologic features [14,15,16,17,18] although its morphology and genome organization is similar to that of other members of the subfamily. Nipah virus has six genes arranged in tandem, 3′-N, P, M, F, G and L-5′ [15,16]. The N, P and L are required for reconstituting viral RNA polymerase activity, the matrix protein M is required for particle formation and budding, and the two surface glycoproteins G and F are required for attachment and entry into the host cell [19,20]. EphrinB2 and B3 have been identified as the Nipah virus entry receptors [21,22,23]. After fusion of the virus and the cell membrane, the viral ribonucleoprotein is released into the cell cytoplasm. Following transcription and replication, the viral components migrate to the plasma membrane for assembly and budding of progeny particles [24,25].

Two vaccination strategies for Nipah virus disease prevention have been explored. A canarypox virus-based vaccine vector approach was effective as veterinary vaccine [26]. The same approach for human use vaccines is undergoing extensive evaluation, largely for HIV and AIDS [27]. A soluble Nipah virus G protein approach has also shown promise [28,29]. However, subunit approaches are generally less effective than particulate immunogens, and can suffer from suboptimal presentation to the immune system [30,31,32]. Immunogenicity in mice to Nipah virus glycoproteins has been reported using two vectored approaches for gene delivery; one using Venezuelan equine encephalitis virus replicons [33] and the other involving inoculation of a mix of two complementing defective vesicular stomatitis virus (VSVΔG) vectors, one for expressing each of the two Nipah virus glycoproteins [34]. The latter approach is new and seems promising but its regulatory approval as human vaccine might be problematic [34,35].

Thus, there is a recognized need in the art for a vaccine for Nipah virus. The present invention fulfills this long-standing need and desire in the art.

### SUMMARY OF THE INVENTION

The present invention is directed to a Paramyxovirus virus-like particles, wherein said virus-like particles are composed of surface glycoprotein G; surface glycoprotein F; and matrix protein M. The present invention is directed further to an expression vector comprising a nucleotide sequence that encodes the virus-like particles of the present invention. The present invention is directed further to a vaccine comprising the virus-like particles of the present invention and a pharmaceutically acceptable carrier or an adjuvant which is well known in the art. The present invention is directed further to a method of vaccinating a subject against paramyxovirus infection comprising administering the vaccine of the present invention.

The present invention demonstrated the potential of Nipah virus virus-like particles (VLPs) as a vaccine. Plasmid-mediated expression of selected viral proteins results in the spontaneous assembly and release of VLPs. These particles make highly effective immunogens because they possess several features of the authentic virus such as their surface structure and dimensions [31,36]. They are also safe because they do not contain any viral genetic material. VLPs, where one or more of the constituent proteins serve as immunogens (native VLPs), can be effective as vaccines for infectious disease. Using this approach for vaccine development, vaccines for human papillomavirus and Hepatitis B virus have been approved for human use, and many, for non-enveloped and enveloped viruses [31,32,37,38,39,40,41,42] are at various stages of development.

The budding capacity of virus proteins as VLPs, the protein-protein interactions that facilitate this process, and the central role of M protein in VLP assembly and release has been described for several paramyxoviruses such as Sendai virus (SeV), Newcastle disease virus (NDV), respiratory syncytial virus (RSV), paramyxovirus simian virus 5 (PIV-5) and human parainfluenza virus type 1 (hPIV1) [20,43,44,45,46,47]. The efficiency of VLP formation in virtually all these studies was based on M protein release in the supernatant. NiV virus-like particles have also been described [48]; the results of this study showed 1) that NiV G and F proteins individually retained some budding capacity although it was far less efficient than that of the M protein and 2) NiV N, M, F and G-containing VLPs resembled the virus in some respects but differed significantly from it with respect to ratio of VLP-incorporated F protein; most of it was present in precursor F₀ form. Recently, the vaccine potential of native VLPs of NDV [49] has been described: these particles, composed of HN, F, M and NP proteins, had several virus-like properties. However, since the F protein in this formulation was modified by design to ablate the cleavage site, it remained in its precursor form; consequently, the NDV VLPs were non-fusogenic, and therefore incapable of inducing syncytia formation.

Other and further objects, features, and advantages will be apparent from the following description of the presently preferred embodiments of the invention, which are given for the purpose of disclosure.

### BRIEF DESCRIPTIONS OF THE DRAWINGS

So that the matter in which the above-recited features, advantages and objects of the invention, as well as others which will become clear, are attained and can be understood in detail, more particular descriptions of the invention briefly summarized above may be had by reference to certain embodiments thereof which are illustrated in the appended drawings. The appended drawings illustrate preferred embodiments of the invention and therefore are not to be limiting in their scope.

FIGS. 1A-1B depict the amount of M, F and G plasmids used at transfection has a bearing on the level of particle formation based on minigenome-encoded reporter gene levels in VLP-infected cells. Cells were transfected with increasing concentrations of M, or F or G expression plasmids (indicated by a triangle) while keeping the concentration of the other two plasmids fixed. They were co-transfected with the previously optimized minigenome and N, P and L constructs [16,57]. Forty eight hours post transfection, the cell SUPS were clarified by centrifugation, and same volume of SUP from each sample was used to infect new cell monolayers (VLP infected) which were transfected 24 hours previously with the core plasmids N, P and L to support replication of the VLP-incorporated minigenome RNA; the VLP-infected cells were harvested 48 hours later for reporter gene analysis. FIG. 1A shows the plasmids transfected in each reaction. FIG. 1B: shows minigenome-encoded CAT activity in VLP infected cell monolayers. Lane 1 is a negative control. Absence of CAT activity in duplicate lanes 2 and 3 indicates that VLP formation, and consequently VLP-incorporated minigenome transfer and expression, does not occur in the absence of M, F and G proteins. The results in lanes 4 through 15 shows that CAT levels varied in VLP infected samples depending on the concentration of M, F and G constructs used at transfection. Thus, the amount of M, F and G plasmids used at transfection had a bearing on the level of particle formation, and the consequent CAT reporter gene transfer and expression. CAT activity in the VLP infected reactions appeared optimal in the boxed lanes 7 and 8. Further analysis of CAT levels in the linear range (data not shown) demonstrated that optimal VLP formation was achieved with the ratios of M, F and G expression plasmids of 3:1:1 (lane 7).

FIGS. 2A-2E show that co-expression of NiV proteins G, F and M results in the formation substantial quantities of VLPs morphologically resembling NiV virions: VLPs released in the transfected cell-supernatant were harvested and purified as described below, and viewed by EM and cryoEM to evaluate their morphology. Under optimized conditions, substantial amounts of VLPs were produced. FIG. 2A: shows VLP-containing band in the sucrose gradient. Negatively stained sample in FIG. 2B show numerous well preserved VLPs. Selected VLPs which were magnified (FIG. 2C) to show clearly the spikes of the glycoproteins present on the VLP surface; an occasional particle had what appeared to be a double fringe (shown with an arrow), a feature normally thought to be associated with Hendra virus particles [9]. FIG. 2D: Shows cryoelectron micrograph of one of the VLPs of the present invention. The glycoprotein spikes and their spatial arrangement are seen here even more clearly. (FIG. 2E) Shows functional assembly and immunoreactivity of NiV glycoproteins at the VLP surface. Unfixed particles were stained by immunogold labeling technique using NiV-specific polyclonal antibody and gold labeled secondary antibody. Unfixed particles were used so that only the surface proteins would be available for immunoreactivity. The Figure shows two VLPs with gold-decorated proteins on the VLP surface.

FIG. 3 shows VLP-incorporated NiV proteins. Western blot analysis of NiV VLPs to verify their composition. The VLPs were processed and analyzed by SDS-PAGE as described using manufacturer's instructions. VLP protein bands corresponding in size to NiV proteins G, F₀, F₁ and M were clearly visible.

FIGS. 4A-4D show NiV VLP-induced syncytia in 293 cells is blocked by prior treatment with NiV-specific antibody. NiV VLPs were pre-incubated for one hour at 37° C. with either NiV specific antibody or Junin virus-(JV) specific antibody, or with OPTI-MEM I medium only (untreated VLPs) before inoculating onto 293 cell monolayers grown overnight in 60 mm dishes. The plates were incubated overnight at 37° C. and stained with crystal violet. The results show VLP-mediated formation of syncytia (FIGS. 4A and 4B) that were blocked (FIG. 4C) when the VLPs were pretreated with NiV-specific antiserum but not blocked (FIG. 4D) when the VLPs were pre-treated with Junin virus-specific antibody. Images e and f show uninfected 293 cells. Arrow points to syncytia.

FIGS. 5A-5B: NiV VLP-induced immune response in Balb/c mice: NiV-specific antibody levels of serum samples from mice immunized subcutaneously three times were measured by IFA and by Bio-Plex microsphere methods. Neutralizing antibody response was evaluated by PRNT₅₀. The experiments were done in duplicate. FIG. 5A: For evaluation by IFA, sera from each treatment group were pooled for analysis. The results show serocoversion for each of the four treatment groups. In general, the titers increased progressively with time and with the VLP dose although by day 35, similar titers were seen with the three higher VLP doses. FIG. 5B: Shows neutralizing antibody titers (PRNT₅₀) in sera from each mouse collected on the stated days. Neutralizing antibodies were seen starting on day 28 after primary inoculation. The response was again clearly dose dependent; all mice in the two highest treatment groups C and D showed neutralizing response by day 35. Such response was seen in 3 of 5 and 1 of 5 mice in the two lower treatment groups B and A, respectively.

FIGS. 6A-6B: VLP-induced modulation in transcription profile of genes involved in signaling of innate immune response in 293 cells by PCR Array. 293 cells were grown overnight in 60 mm dishes and were infected with 10 μg of purified VLPs suspended in OPTI-MEM (Invitrogen). Mock infected cells served as negative control. The inoculum was adsorbed on the cell monolayers for 3 hours at 37° C. when it was supplemented with fresh OPTI-MEM and further incubated overnight when total cell RNA was extracted according to the manufacturer's (SA Biosciences) instructions. FIG. 6A: shows the integrity of the RNA used for this analysis. Note the ˜2:1 ratio of 28S:18S which is a good indication of the integrity of the RNA. Equal concentration of the RNA from the mock and VLP-exposed cells was used for expression profiling by RT² PCR Profiler PCR Array according the manufacturer's (SABiosciences) instructions. FIG. 6B: Shows the heat map, it is a visual illustration of the relative expression levels in the VLP-stimulated vs. the “mock” stimulated control cells of the all the genes in the array: The four genes differentially expressed by a factor of ˜4 fold or greater (shown as red squares) are listed below the heat map.

FIGS. 7A-7D shows the morphology of NiVgfmn VLps, and NiVgfmnpl VLPs when viewed by electron microscopy, and their ability to induce syncytia formation, i.e., their fusogenicity in 293 cell monolayers. FIGS. 7A-7BB shows VLPs composed of G, F, M and N proteins (NiVgfmn VLps) and FIGS. 7C-7D shows VLPs composed of G, F, M, N, P and L proteins of the virus (NiVgfmnpl VLPs). When viewed by electron microscopy (image on the left in FIGS. 7A and 7C), both these particles appear to be distinct morphologically to some extent from each other, and from the G, F and M protein-containing VLPs as described in FIG. 2. As expected, VLPs of all three compositions retain their fusogenicity (right hand image in each panel of FIGS. 7B-7D) as shown in FIG. 4.

### DETAILED DESCRIPTION OF THE INVENTION

The present invention describe the vaccine potential of NiV virus-like particles (NiV VLPs) composed of three NiV proteins G, F and M. Co-expression of these proteins under optimized conditions resulted in quantifiable amounts of VLPs with many virus-like/vaccine desirable properties including some not previously described for VLPs of any paramyxovirus: The particles were fusogenic, inducing syncytia formation; PCR array analysis showed NiV VLP-induced activation of innate immune defense pathways; the surface structure of NiV VLPs imaged by cryoelectron microscopy was dense, ordered, and repetitive, and consistent with similarly derived structure of paramyxovirus measles virus. The VLPs were composed of all the three viral proteins as designed, and their intracellular processing also appeared similar to NiV virions. The size, morphology and surface composition of the VLPs were consistent with the parental virus, and importantly, they retained their antigenic potential. These particles, formulated without adjuvant, were able to induce neutralizing antibody response in Balb/c mice. These findings indicate vaccine potential of these particles and will be the basis for undertaking protective efficacy studies in animal models of NiV disease.

The practice of the present invention will employ, unless otherwise indicated, conventional methods of chemistry, biochemistry, molecular biology, immunology and pharmacology, within the skill of the art. Such techniques are explained fully in the literature. See, e.g., Remington's Pharmaceutical Sciences, 18th Edition (Easton, Pa.: Mack Publishing Company, 1990); Methods In Enzymology (S. Colowick and N. Kaplan, eds., Academic Press, Inc.); and Handbook of Experimental Immunology, Vols. I-IV (D. M. Weir and C. C. Blackwell, eds., 1986, Blackwell Scientific Publications); Sambrook, et al., Molecular Cloning: A Laboratory Manual (2nd Edition, 1989); Handbook of Surface and Colloidal Chemistry (Birdi, K. S. ed., CRC Press, 1997); Short Protocols in Molecular Biology, 4th ed. (Ausubel et al. eds., 1999, John Wiley & Sons); Molecular Biology Techniques: An Intensive Laboratory Course (Ream et al., eds., 1998, Academic Press); PCR (Introduction to Biotechniques Series), 2nd ed. (Newton & Graham eds., 1997, Springer Verlag); Peters and Dalrymple, Fields Virology, 2nd ed., Fields et al. (eds.) (B.N. Raven Press, New York, N.Y.).

As used herein the specification, “a” or “an” may mean one or more. As used herein in the claim(s), when used in conjunction with the word “comprising”, the words “a” or “an” may mean one or more than one. As used herein “another” or “other” may mean at least a second or more of the same or different claim element or components thereof. Similarly, the word “or” is intended to include “and” unless the context clearly indicates otherwise. “Comprise” means “include.” It is further to be understood that all base sizes or amino acid sizes, and all molecular weight or molecular mass values, given for nucleic acids or polypeptides are approximate, and are provided for description. Although methods and materials similar or equivalents to those described herein can be used in the practice or testing of the present disclosure, suitable methods and materials are described below. In case of conflict, the present specification, including explanations of terms, will control. In addition, the materials, methods, and examples are illustrative only and not intended to be limiting. Furthermore, unless otherwise required by context, singular terms shall include pluralities and plural terms shall include the singular.

The term “antigen” as used herein is defined as a compound, composition, or substance that can stimulate the production of antibodies or a T cell response in an animal, including compositions that are injected or absorbed into an animal. An antigen reacts with the products of specific humoral or cellular immunity, including those induced by heterologous immunogens. The term “antigen” includes all related antigenic epitopes. “Epitope” or “antigenic determinant” refers to a site on an antigen to which B and/or T cells respond. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance.

An “antigen” can be a tissue-specific antigen, or a disease-specific antigen. These terms are not exclusive, as a tissue-specific antigen can also be a disease specific antigen. A tissue-specific antigen is expressed in a limited number of tissues, such as a single tissue. Specific, non-limiting examples of a tissue specific antigen are a prostate specific antigen. A disease-specific antigen is expressed coincidentally with a disease process. Specific non-limiting examples of a disease-specific antigen are an antigen whose expression correlates with, or is predictive of, tumor formation, such as prostate cancer. A disease specific antigen may be an antigen recognized by T cells or B cells.

The term “antibody” as used herein includes Immunoglobulin molecules and immunologically active portions of immunoglobulin molecules, i.e., molecules that contain an antigen binding site that specifically binds (immunoreacts with) an antigen. A naturally occurring antibody (e.g., IgG, IgM, IgD) includes four polypeptide chains, two heavy (H) chains and two light (L) chains interconnected by disulfide bonds. However, it has been shown that the antigen-binding function of an antibody can be performed by fragments of a naturally occurring antibody. Thus, these antigen-binding fragments are also intended to be designated by the term “antibody.” Specific, non-limiting examples of binding fragments encompassed within the term antibody include (i) a Fab fragment consisting of the V_{L}, V_{H}, C_{L} and C_{H1} domains; (ii) an F_{d} fragment consisting of the V_{H} and C_{H1} domains; (iii) an Fv fragment consisting of the V_{L} and V_{H} domains of a single arm of an antibody, (iv) a dAb fragment (Ward et al., Nature 341:544-546, 1989) which consists of a V_{H} domain; (v) an isolated complimentarily determining region (CDR); and (vi) a F(ab′)₂ fragment, a bivalent fragment comprising two Fab fragments linked by a disulfide bridge at the hinge region.

Immunoglobulins and certain variants thereof are known and many have been prepared in recombinant cell culture (e.g., see U.S. Pat. No. 4,745,055; U.S. Pat. No. 4,444,487; WO 88/03565; EP 256,654; EP 120,694; EP 125,023; Faoulkner et al., Nature 298:286, 1982; Morrison, J. Immunol. 123:793, 1979; Morrison et al., Ann Rev. Immunol 2:239, 1984).

The term “subject” or “animal” as used herein refers to living multi-cellular vertebrate organisms, a category that includes, for example, mammals and birds. The term mammal includes both human and non-human mammals. Similarly, the term “subject” includes both human and veterinary subjects.

The term conservative variation includes the use of a substituted amino acid in place of an unsubstituted parent amino acid, provided that antibodies raised to the substituted polypeptide also immunoreact with the unsubstituted polypeptide. Non-conservative substitutions are those that reduce an activity or antigenicity.

The term “cDNA” (complementary DNA) refers to a piece of DNA lacking internal, non-coding segments (introns) and regulatory sequences that determine transcription. cDNA is synthesized in the laboratory by reverse transcription from messenger RNA extracted from cells.

The term “diagnostic” refers to identifying the presence or nature of a pathologic condition, such as, but not limited to, prostate cancer. Diagnostic methods differ in their sensitivity and specificity. The “sensitivity” of a diagnostic assay is the percentage of diseased individuals who test positive (percent of true positives). The “specificity” of a diagnostic assay is 1 minus the false positive rate, where the false positive rate is defined as the proportion of those without the disease who test positive. While a particular diagnostic method may not provide a definitive diagnosis of a condition, it suffices if the method provides a positive indication that aids in diagnosis. “Prognostic” is the probability of development (e.g., severity) of a pathologic condition, such as prostate cancer, or metastasis.

An “epitope” as used herein, is an antigenic determinant. These are particular chemical groups or peptide sequences on a molecule that are antigenic, i.e. that elicit a specific immune response. An antibody specifically binds a particular antigenic epitope on a polypeptide. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, Glenn E. Morris, Ed (1996).

The term “expression control sequence” refers to Nucleic acid sequences that regulate the expression of a heterologous nucleic acid sequence to which it is operatively linked. Expression control sequences are operatively linked to a nucleic acid sequence when the expression control sequences control and regulate the transcription and, as appropriate, translation of the nucleic acid sequence. Thus expression control sequences can include appropriate promoters, enhancers, transcription terminators, a start codon (i.e. ATG) in front of a protein-encoding gene, splicing signal for introns, maintenance of the correct reading frame of that gene to permit proper translation of mRNA, and stop codons. The term “control sequences” is intended to include, at a minimum, components whose presence can influence expression, and can also include additional components whose presence is advantageous, for example, leader sequences and fusion partner sequences. Expression control sequences can include a promoter.

The term “promoter” refers to a minimal sequence sufficient to direct transcription. Also included are those promoter elements which are sufficient to render promoter-dependent gene expression controllable for cell-type specific, tissue-specific, or inducible by external signals or agents; such elements may be located in the 5′ or 3′ regions of the gene. Both constitutive and inducible promoters are included (see e.g., Bitter et al., Methods in Enzymology 153:516-544, 1987). For example, when cloning in bacterial systems, inducible promoters such as pL of bacteriophage lambda, plac, ptrp, ptac (ptrp-lac hybrid promoter) and the like may be used. In one embodiment, when cloning in mammalian cell systems, promoters derived from the genome of mammalian cells (e.g., metallothionein promoter) or from mammalian viruses (e.g., the retrovirus long terminal repeat; the adenovirus late promoter; the vaccinia virus 7.5K promoter) can be used. Promoters produced by recombinant DNA or synthetic techniques may also be used to provide for transcription of the nucleic acid sequences.

As defined herein, the term “host cell” refers to cells in which a vector can be propagated and its DNA expressed. The cell may be prokaryotic or eukaryotic. The term also includes any progeny of the subject host cell. It is understood that all progeny may not be identical to the parental cell since there may be mutations that occur during replication. However, such progeny are included when the term “host cell” is used.

The term “immune response” refers to a response of a cell of the immune system, such as a B cell, T cell, or monocyte, to a stimulus. In one embodiment, the response is specific for a particular antigen (an “antigen-specific response”). In one embodiment, an immune response is a T cell response, such as a CD4+ response or a CD8+ response. In another embodiment, the response is a B cell response, and results in the production of specific antibodies.

An “isolated” biological component (such as a nucleic acid or protein or organelle) as defined herein, has been substantially separated or purified away from other biological components in the cell of the organism in which the component naturally occurs, i.e., other chromosomal and extra-chromosomal DNA and RNA, proteins and organelles. Nucleic acids and proteins that have been “isolated” include nucleic acids and proteins purified by standard purification methods. The term also embraces nucleic acids and proteins prepared by recombinant expression in a host cell as well as chemically synthesized nucleic acids.

A “label” as defined herein, is a detectable compound or composition that is conjugated directly or indirectly to another molecule to facilitate detection of that molecule. Specific, non-limiting examples of labels include fluorescent tags, enzymatic linkages, and radioactive isotopes.

The term “oligonucleotide” refers to a linear polynucleotide sequence of up to about 100 nucleotide bases in length.

Open reading frame (ORF) is defined as a series of nucleotide triplets (codons) coding for amino acids without any internal termination codons. These sequences are usually translatable into a peptide.

The term “operably linked” refers to a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

As used herein, the term “vector” refers to a nucleic acid molecule as introduced into a host cell, thereby producing a transformed host cell. A vector may include nucleic acid sequences that permit it to replicate in a host cell, such as an origin of replication. A vector may also include one or more selectable marker genes and other genetic elements known in the art.

As used herein, the term “transduction” encompasses all techniques by which a nucleic acid molecule might be introduced into such a cell, including transfection with viral vectors, transformation with plasmid vectors, and introduction of naked DNA by electroporation, lipofection, and particle gun acceleration. A transduced cell is a cell into which has been introduced a nucleic acid molecule by molecular biology techniques.

As used herein, the term “T Cell” refers to a white blood cell critical to the immune response. T cells include, but are not limited to, CD4⁺ T cells and CD8⁺ T cells. A CD4⁺ T lymphocyte is an immune cell that carries a marker on its surface known as “cluster of differentiation 4” (CD4). These cells, also known as helper T cells, help orchestrate the immune response, including antibody responses as well as killer T cell responses. CD8⁺ T cells carry the “cluster of differentiation 8” (CD8) marker. In one embodiment, a CD8 T cell is a cytotoxic T lymphocyte. In another embodiment, a CD8 cell is a suppressor T cell.

A “recombinant nucleic acid” is one that has a sequence that is not naturally occurring or has a sequence that is made by an artificial combination of two otherwise separated segments of sequence. This artificial combination is often accomplished by chemical synthesis or, more commonly, by the artificial manipulation of isolated segments of nucleic acids, e.g., by genetic engineering techniques.

The term “polynucleotide” or “nucleic acid sequence” refers to a polymeric form of nucleotide at least 10 bases in length. A recombinant polynucleotide includes a polynucleotide that is not immediately contiguous with both of the coding sequences with which it is immediately contiguous (one on the 5′ end and one on the 3′ end) in the naturally occurring genome of the organism from which it is derived. The term therefore includes, for example, a recombinant DNA which is incorporated into a vector; into an autonomously replicating plasmid or virus; or into the genomic DNA of a prokaryote or eukaryote, or which exists as a separate molecule (e.g., a cDNA) independent of other sequences. The nucleotides can be ribonucleotides, deoxyribonucleotides, or modified forms of either nucleotide. The term includes single- and double-stranded forms of DNA.

The term peptide, as used herein refers to any chain of amino acids, regardless of length or post-translational modification (e.g., glycosylation or phosphorylation).

The term “probe” comprises an isolated nucleic acid attached to a detectable label or reporter molecule. The term “primer” includes short nucleic acids, preferably DNA oligonucleotides, 15 nucleotides or more in length. Primers may be annealed to a complementary target DNA strand by nucleic acid hybridization to form a hybrid between the primer and the target DNA strand, and then extended along the target DNA strand by a DNA polymerase enzyme. Primer pairs can be used for amplification of a nucleic acid sequence, e.g., by the polymerase chain reaction (PCR) or other nucleic-acid amplification methods known in the art. One of skill in the art will appreciate that the specificity of a particular probe or primer increases with its length. Thus, for example, a primer comprising 20 consecutive nucleotides will anneal to a target with a higher specificity than a corresponding primer of only 15 nucleotides. Thus, in order to obtain greater specificity, probes and primers may be selected that comprise 20, 25, 30, 35, 40, 50 or more consecutive nucleotides.

The term “promoter” as described herein, is an array of nucleic acid control sequences that directs transcription of a nucleic acid. A promoter includes necessary nucleic acid sequences near the start site of transcription, such as, in the case of a polymerase II type promoter, a TATA element. A promoter also optionally includes distal enhancer or repressor elements which can be located as much as several thousand base pairs from the start site of transcription. Both constitutive and inducible promoters are included (see e.g., Bitter et al., Methods in Enzymology 153:516-544, 1987). Specific, non-limiting examples of promoters include promoters derived from the genome of mammalian cells (e.g., metallothionein promoter) or from mammalian viruses (e.g., the retrovirus long terminal repeat; the adenovirus late promoter; the vaccinia virus 7.5K promoter) may be used. Promoters produced by recombinant DNA or synthetic techniques may also be used. A polynucleotide can be inserted into an expression vector that contains a promoter sequence which facilitates the efficient transcription of the inserted genetic sequence of the host. The expression vector typically contains an origin of replication, a promoter, as well as specific nucleic acid sequences that allow phenotypic selection of the transformed cells.

Pharmaceutically acceptable carriers: The pharmaceutically acceptable carriers of use are conventional. Remington's Pharmaceutical Sciences, by E. W. Martin, Mack Publishing Co., Easton, Pa., 15th Edition (1975), describes compositions and formulations suitable for pharmaceutical delivery of the vaccines herein disclosed.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

As used herein, the term “subject” refers to any target of the treatment. Preferably, the subject is a mammal, more preferably, the subject is a canine or a human.

In one embodiment of the present invention, there are provided paramyxovirus virus-like particles, wherein said virus-like particles are composed of surface glycoprotein G; surface glycoprotein F; and matrix protein M. The paramyxovirus virus-like particles may be prepared from any paramyxovirus included but not limited to Avulavirus, Henipavirus, Morbillivirus, Respirovirus, Rubulavirus, TPMV-like viruses, Pneumovirinae, Pneumovirus and Metapneumovirus. In one preferred embodiment, the paramyxovirus virus-like particles are from Henipavirus and are from Nipah Virus. Generally, for these paramyxovirus virus-like particles, the G and F proteins mediate attachment and entry into the host cell. These paramyxovirus virus-like particles activate innate immune signaling in cells infected with paramyxovirus Virus. These virus-like particles induce robust neutralizing antibody response. Importantly, the paramyxovirus virus-like particles of the present invention are fusogenic and induce syncytia formation. The paramyxovirus virus-like particles may have biologically active G and F proteins on the particle surface and can be purified.

In another embodiment of the present invention, there is provided an expression vector comprising a nucleotide sequence that encodes the virus-like particles described above. In another embodiment of the present invention, there is provided a vaccine comprising the virus-like particles described above and a pharmaceutically acceptable carrier.

In another embodiment of the present invention, there is provided a method of vaccinating a subject against paramyxovirus infection comprising administering the vaccine of described above. The method may be applied to any subject including but not limited to a human or animals. A person having ordinary would readily recognize that the virus-like particles of the present invention may be administered by a variety of routes, including but not limited to intramuscular, subcutaneous or any other as known in the art. Further, the administering may be in a single dose or multiple doses or as needed to achieve the desired effect. Preferably, the particles are administered comprises about 5 micrograms to about 200 micrograms of said virus-like particles.

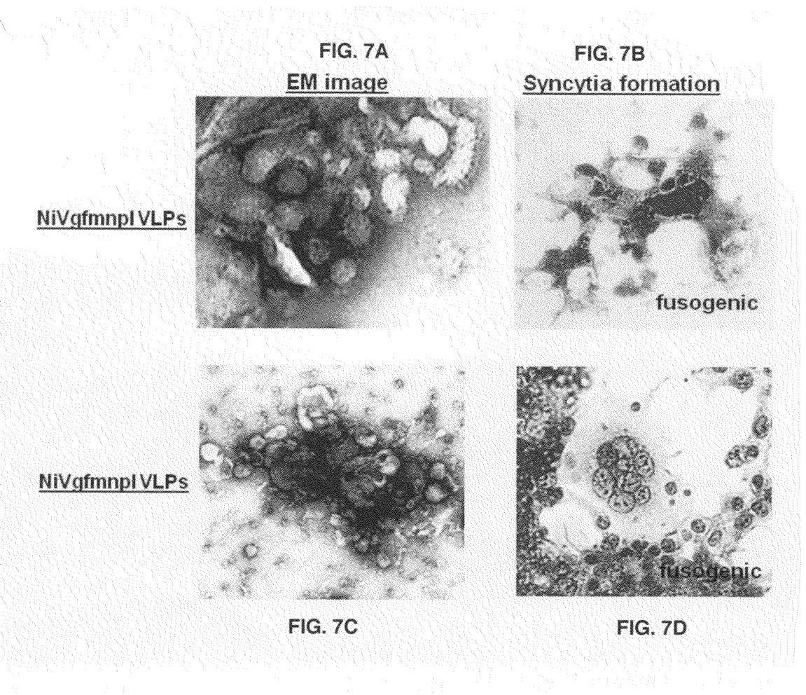

In another embodiment of the present invention, there are provided different VLP compositions and fusogenicity. The present invention demonstrated the vaccine potential of NiV virus-like particles composed of the two viral surface glycoproteins G and F, and the matrix protein M (NiVgfmn VLPs as well as those made out of N, P and L proteins in addition to G, F and N proteins, i.e., NiVmfgnpl VLPs. Both these VLP compositions produce fusogenic particles (see FIG. 7). This is the minimal essential composition to make particles that resemble the real virus morphologically and immunologically. In paramyxoviruses, the two surface glycoproteins are required for attachment and entry into the host cell. The F protein, which is synthesized as an inactive precursor F0 form has to undergo post-translational proteolytic processing to generate two disulfide-linked subunits F1 and F2, thereby releasing the hydrophobic fusion peptide located at the amino terminus of F1. This process is essential for the generation of a mature and functional fusogenic form that mediates virus-host cell interaction and entry into the host cell. It also mediates cell-cell fusion and syncytia formation. In the real virus, and in the VLPs of the present invention, there is substantial amount fusogenic F1 form and this may be critically important in conferring fusogenicity and vaccine potency. The G and F proteins are the major source of neutralizing antibody response, together they induce the highest neutralizing antibody response, and protection. M protein is required for budding and morphogenesis. The present invention also encompasses VLPs made of additional viral components. This would allow understanding of properties of VLPs composed not only of surface glycoproteins but of additional viral components (such as promoter regions, and some of the internal proteins) which, in some paramyxoviruses like measles virus, are known to modulate host innate immune responses, and may thus impact on type and duration of protection conferred by a VLP vaccine. For example, the present invention provides VLPs composed of N protein in addition to the G, F and M proteins, i.e., NiVmfgn VLPs as well as NiVmfgnpl VLPs. Both these VLP compositions produce fusogenic particles that are (see attached Figure). For example, the present invention includes (1) recombinant virus-like particles wherein said the protein of interest additionally comprises of the N protein and/or L and/or P protein of the virus; (2) recombinant virus-like particles (VLPs) of claim 1, wherein said protein of interest additionally comprises of the C and/or W proteins of the virus; (3) recombinant virus-like particles wherein the protein of interest additionally comprises any or all other viral proteins; (4) recombinant virus like particles of any composition encapsidating synthetic RNAs. The RNA incorporates viral promoter region sequences at their termini similarly to that in the viral genomic RNA. The present invention includes fusogenic VLPs made by any viral protein delivery technique in cells of mammalian or any other origin, whether adherent or non-adherent, whether adherent or in suspension. Examples are transient transfection of protein expression plasmids, stably transfected inducible cell lines, baculovirus expression system, lentivirus expression system and others.

The present invention also envisions that the ratios of G:F:M proteins and F0:F1 proteins may be manipulated for optimum results. For example, in the paramyxovirus virus-like particles of the present invention, G may be in the range of 0.7 to 1.3 and F may be in the range of 0.7 to 1.3 and M in the range of 2.6 to 3.4. In another aspect, G may be in the range of 0.8 to 1.2 and F may be in the range of 0.8 to 1.2 and M in the range of 2.7 to 3.3. In another aspect, G may be in the range of 0.9 to 1.1 and F may be in the range of 0.9 to 1.1 and M may be in the range of 2.8 to 3.2. In yet another aspect, G may be in the range of 0.9 to 1.1 and F may be in the range of 0.9 to 1.1 and M may be in the range of 2.9 to 3.1. Similarly, the range of F0, F1 and F1 may be varied, including but not limited to (1) F1 is in the range 40-60% and F0 in the range 60-40% with F2, F1 and F0 adding up to 100%; (2) F1 in the range 30-70% and F0 in the range 70-30% with F2, F1 and F2 adding up to 100%; (3) F1 in the range 20-80% and F0 in the range 80-20% with F2, F1 and F2 adding up to 100%; and (4) F1 is in the range 10-90% and F0 in the range 10-90% with F2, F1 and F0 adding up to 100%.

The following examples are given for the purpose of illustrating various embodiments of the invention and are not meant to limit the present invention in any fashion.

The present invention describes NiV VLPs composed of the two surface glycoproteins G, and F, and the matrix protein M. The G and F proteins were included because they mediate attachment and entry into the host cell [50,51,52], both are major targets of neutralizing antibodies, and both are major players in vaccine induced protection [52,53,54]. NiV G and F together are also the most effective as immunogens; this was elucidated in a canary pox virus vector-based experimental protective efficacy study [26]. The M protein was included in the formulation because it is required for particle formation and release [20,25,45]. Under optimized conditions, substantial, quantifiable amounts of NiV VLPs composed of these three NiV proteins were made. This has allowed characterization of their properties in detail to show that they possessed many virus-like/vaccine desirable properties in vitro. It has also allowed testing for immunogenicity in vivo in Balb/c mice; note that although NiV does not cause disease in these animals, NiV proteins injected in them are known to induce robust neutralizing antibody response [29,33,34]. Importantly, NiV-specific mouse monoclonal antibodies are protective in the hamster model of NiV disease [55].

In this study, careful assessment of immunogenicity has shown for the first time, that these NiV VLPs are able to induce neutralizing antibody response. A detailed methodology is also provided to optimize production of the VLPs for research purposes. Beyond this, also provided is the first CryoEM study of NiV VLPs and a careful assessment of their morphology. NiV VLPs can trigger “fusion from without” upon addition to cells, a first for an enveloped VLP. Finally, it is shown that NiV VLPs activate innate immune signaling in “infected” cells and provide a transcriptional profile of this response. Based on all these attributes, NiV M, F and G-protein-containing VLPs show promise as vaccine and will be the basis for undertaking protective efficacy studies in animal models of NiV disease.

### EXAMPLE 1

### Protein Expression Vectors, Cells and Viruses

NiV expression plasmids pCAGGS-G, F, and M were all under the control of chicken beta actin promoter [56], and were constructed as described [20]. Human embryonic kidney 293 cells (ATCC, CRL-1573) and 293T cells (ATCC, CRL-11268) were grown in Dulbecco's minimum essential medium supplemented with 10% fetal bovine serum (FBS) and penicillin and streptomycin, and maintained in the same medium containing 2% FBS. The minigenome was used for optimizing VLP formation as described [57]. All the initial minigenome-based optimization steps were done in BHK-T7 cells (obtained from Dr. N. Ito). These conditions were applicable to produce VLPs in 293T cells and were used throughout to generate the VLPs described herein.

### EXAMPLE 2

### Transfection

293T cells were grown in Dulbecco's complete medium to achieve semi-confluent (80-90% density) cell monolayers. The cells were transiently transfected with the plasmids constructs using the lipid reagent Lipofectamine 2000 according to the guidelines provided by the manufacturers' instructions (Invitrogen Inc). At 48 hrs post-transfection, the VLP-containing cell supernatants (SUP) were harvested for concentration and purification of the VLPs. Because of the fusogenic property of these VLPs, there was widespread syncytia formation at this time point although the cells were still adherent.

### EXAMPLE 3

### VLP Harvest and Purification

VLPs released in the transfected-cell SUP were harvested and clarified by centrifugation at 3,500 rpm for 30 minutes at 4° C. and concentrated by sucrose density gradient centrifugation based on previous descriptions [44,45,58]. Briefly, the clarified SUPs were concentrated by ultracentrifugation through 20% sucrose cushion in TN buffer (0.1M NaCl; 0.05M Tris-HCL, pH 7.4) at 200,000×g for 8 hours at 4° C. The resulting VLP pellet in ˜0.5 ml volume was purified on a discontinuous sucrose gradient formed by layering 80%, 65%, 50% and 10% sucrose in TN buffer. After centrifugation at 186,000×g for 8 hours, the top ˜1.5 ml of the gradient (which included the VLP-containing band at the interface between the 10% and 50% sucrose layers) was resuspended in 20% sucrose buffer and centrifuged once more at 160,000×g for one hour. The resulting pellet was resuspended in 20% sucrose solution in endotoxin-free TN bufffer and stored at 4° C. for subsequent analysis. Supernatant of 293T cells transfected with empty pCAGGS plasmid and processed similarly (referred to as “mock” particles) served as negative control when needed.

### EXAMPLE 4

### VLP Infectivity Assay

Since the ratio of the protein expression plasmids used at transfection and the time of harvest may have a bearing on the level of VLP formation, a minigenome-based VLP infectivity assay, similar to those described previously [59,60] was used to determine the relative concentrations of the constituent plasmids, and to determine the kinetics of VLP formation for optimal production. This assay provides only a comparative assessment of VLP formation since it only accounts for VLPs that are able to incorporate and passage minigenomes. However, based on the assumption that the ratio of empty and minigenome-containing VLPs will be equivalent in each reaction, the method provides an indirect means to determine the optimal set of conditions for VLP production as determined by VLP-incorporated minigenome-encoded CAT enzyme activity. Briefly, the steps involved in the VLP infectivity assay were 1) transfection of NiV minigenome construct and co-transfection with full complement of the NiV protein expression plasmids, N, P, L, M, F and G, using Lipofectamine 2000. 2) following replication (48 hours post-transfection), passage of equal volume of VLP-containing transfected cell SUP on to fresh cells previously transfected with N, P and L plasmids and 3), determination of CAT activity in the VLP infected cells 48 hours later. Replication of the VLP-incorporated incoming minigenomes based on reporter gene activity indicates the level of particle formation and release, VLP infectivity, and successful minigenome packaging.

### EXAMPLE 5

### CAT Assays

FAST CAT Assay kit (Molecular Probes) was used according the manufacturer's instructions and allowed accurate quantification of CAT enzyme levels over a wide linear range.

### EXAMPLE 6

### Electron Microscopy (EM)

VLPs were purified as described. The particles were adsorbed on Formvar carbon coated copper grid by floating it on a drop of VLP suspension for 15 minutes, the grids were blotted, and then negatively stained with 2% aqueous uranyl acetate for viewing by transmission electron microscopy.

### EXAMPLE 7

### Cryoelectron (CryoEM) Microscopy

The VLPs were vitrified as reported [61] on holey carbon film grids (C-flat™, Protochips, Raleigh, N.C.). VLPs were imaged at 40,000× indicated magnification using a 4 k×4 k slow-scan CCD camera (UltraScan 895, GATAN, Inc., Pleasanton, Calif.) using a low-dose imaging procedure.

### EXAMPLE 8

### Immunogold Labeling

Unfixed VLPs were used for immunogold labeling to limit antibody reactivity to the cell surface proteins. The particles were adsorbed on formvar coated nickel grids, stained with NiV specific primary antibody (hyper immune mouse ascites fluid, HMAF, obtained from Dr. P. Rollin, CDC) diluted in buffer (1% BSA in 0.05M tris buffer) rinsed in wash buffer (0.1% BSA in 0.05M tris buffer), stained with colloidal gold labeled goat anti-mouse secondary antibody (Jackson ImmunoResearch Laboratories), washed, and then negatively stained with 2% uranyl acetate for viewing by EM.

### EXAMPLE 9

### VLP Protein Concentration

The total protein concentration of the purified VLP preparations was measured by the BCA (Bicinchoninic Acid) method (Thermo Scientific Laboratories).

### EXAMPLE 10

### Western Blotting

VLP composition was determined by western blot analysis. Briefly, purified VLPs resuspended in endotoxin free PBS were lysed by resuspending them in equal amount of 2×SDS protein-loading buffer and loaded into a 12% SDS-polyacrylamide gel with a 4% stacking gel. 293T cell lysates processed similarly were run in parallel as negative cell control. Following electrophoresis to resolve the protein bands, and transfer to membrane, the blot was incubated with NiV-specific HMAF primary antibody at a dilution of 1:1000 dilution, overnight at 4° C., and HRP-conjugated anti-mouse secondary antibody (from GE Healthcare) at a 1:20,000 dilution for one hour at room temperature. The proteins were revealed using western blot detection reagents according to instructions provided by the manufacturer (GE Healthcare).

### EXAMPLE 11

### Protocol to Immunize Balb/c Mice

These studies were undertaken with the approval of the Institutional Biosafety Committee (Protocol# #01/08-2010-1) and the Institutional Animal Care and Use (IACUC) Committee (Protocol #0904028). Five to six week old female Balb/c mice (Harlan Laboratories) were housed in microisolater cage for 4 days before beginning the immunization protocol. Mice in groups of five were immunized by subcutaneous inoculation of four different concentrations of VLPs (1.75, 3.5, 7 or 14 μg/mouse, referred to subsequently as treatment groups A through D respectively) prepared just prior to use in sterile endotoxin free PBS. No adjuvant was used. A group of five mice inoculated with sterile endotoxin free PBS served as negative control group. Mice in the four treatment groups (A through D) were boosted (6pg/mouse) on days 15 and 29; the negative control group received PBS. Blood was collected from the submandibular vein of the animals on days −1, 14, 21, 28 and 35; they were euthanized on day 35.

### EXAMPLE 12

### Plaque Reduction Neutralization Test (PRNT)

Two-fold dilutions of test sera were made in 50 μl cell culture medium. Under biohazard level 4 conditions, each of the diluted sera were mixed with 50 μl of NiV diluted to generate ˜30 plaque forming units and incubated for 30 min at 37° C. The pre-incubated virus-antibody mix was added to Vero cell monolayers grown in 96 well plates and incubated for 30 min at 37° C. when the inoculum was removed and replaced 150 μl of cell media. After incubation at 37° C. for 24 h, the cells fixed in 100% ice-cold methanol and staining by indirect immunofluorescence assay as follows: The wells in the plate were blocked with BSA/PBS and stained with rabbit sera raised against the G protein of HeV, and goat anti-rabbit Alexa Fluor 488 conjugate (Invitrogen) diluted 1:1000 in blocking buffer. Viral plaques were visualized and counted, and neutralizing antibody titers were reported based on reduction in plaque count by 50% relative to the untreated control (PRNT₅₀).

### EXAMPLE 13

### Antibody Levels Measured by Immunofluorescence Assay (IFA)

For IFA, NiV-specific total antibody levels were measured by using NiV G, F and M expressing 293T cells as target antigen. Thirty six hours post-transfection, the cells were harvested, fixed in paraformaldehyde, cytospun (Cytocentrifuge, Thermoscientific) on glass slides to obtain monolayered preparations and then stored at 4° C., and used as antigen within three weeks of preparation. On the day of use, the slides were washed in PBS, permeabilized with Triton-X-100 and blocked with BSA/PBS. After incubation with two fold dilutions of the test sera, the cell monolayers were washed and stained with Alexa fluor 488-conjugated goat anti-mouse antibody according the manufacturer's (Molecular Probes) instructions. Negative and positive controls were run in parallel with each batch.

### EXAMPLE 14

### Gene Expression Profile by Real-Time PCR

VLP-mediated transcriptional activation was tested for eighty four genes involved in Toll-like receptor (TLR)-mediated signal transduction using RT² Profiler PCR array (SABiosciences). The 96 well array format included mediators of TLR signaling including adaptors and proteins that interact with the TLRs, and members of NFKB, JNKp38, NF/IL6 and IRF signaling pathways downstream of TLR signaling. Briefly, 293 cells grown overnight in 60 mm dishes were exposed to 10 μg of purified VLPs suspended in 1 ml of OPTI-MEM (Invitrogen). “Mock particles” (see, VLP harvest and purification) resuspended similarly and exposed to 293 cells served as negative control. The inoculums were adsorbed on the cell monolayers for 3 hours at 37° C. when additional 1.5 ml of OPTI-MEM was added and the dishes further incubated. Twenty fours post VLP exposure, total cell RNA was extracted according to the manufacturer's (SABiosciences) instructions. The integrity of the RNA was verified by agarose gel electrophoresis and the same concentration of total cell RNA from the VLP-stimulated and “mock” stimulated cells were used for gene expression profiling by Real-time PCR using Eppendorf Mastercycler unit. The array plate included positive and negative controls for quality assurance, and three sets of housekeeping genes for normalization for data analysis. The fold-change in gene expression in the VLP stimulated 293 cells relative to the “mock” stimulated 293 cells was calculated by the _Ct method according to the manufacturer's instructions.

### EXAMPLE 15

### Optimization of Conditions for the Production of NiV VLPs

Co-expression of NiV G, F and M proteins in 293T cells resulted in the formation of VLPs that bud out into the transfected cell SUP and can be harvested, concentrated and purified as described above. However, the VLP yield was low. To improve the efficiency of VLP formation, the ratio of the three expression plasmids used at transfection was optimized. It was speculated that this would be important based on the fact that a), during replication, paramyxoviruses form a transcription gradient where the 3′ proximal genes are transcribed more abundantly than the successive downstream genes [52] and b), the stoichiometry of interaction of the viral proteins has proved to be critical in plasmid-driven minigenome and full-length rescue systems [62].

The importance of protein ratios for VLP formation was alluded to in a previous NDV study where the expression plasmids were co-transfected at “pre-determined concentrations” to produce VLP-incorporated protein ratios analogous to those in virus infected cells [49]. In a study by Patch et al [48], equivalent amounts of NiV N, M, F and G were initially used to produce the VLPs. In that study, VLPs were subsequently also made by adjusting NiV expression plasmid concentrations by experimental variations similarly to that in the NDV study [49]. The efficiency of particle formation and budding in both these, and many other paramyxovirus VLP formation systems was based on M protein release [43,44,45,46,48].

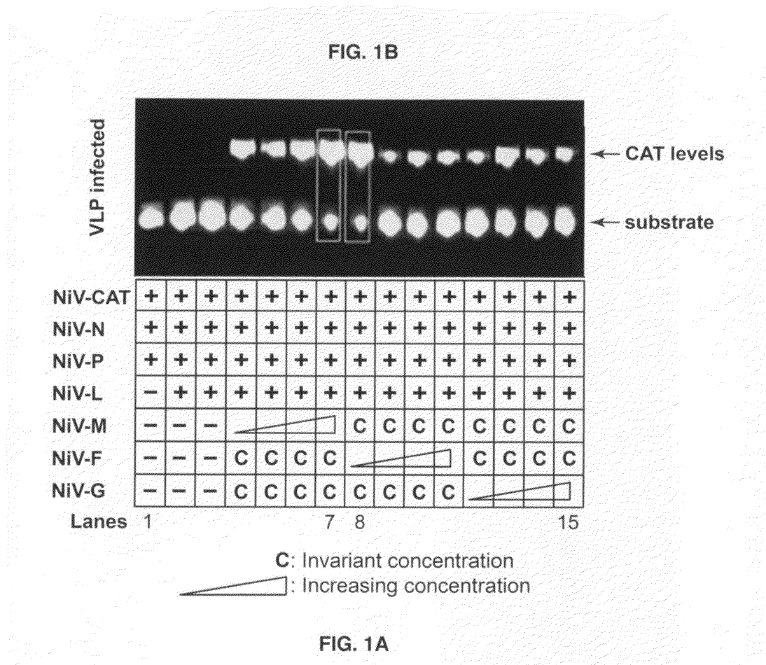

The present invention used a minigenome-based functional assay, the VLP infectivity assay (described above), to determine optimal expression plasmid ratios for efficient VLP formation based on reporter gene readouts. Briefly, 293T cells were transfected with plasmids as shown in FIG. 1. For titrating NiV G, F and M plasmids, increasing concentrations of either G, or F or M expression plasmids were, in turn, co-transfected with fixed concentrations of the other two plasmids. The minigenome and N, P and L plasmids were transfected using a predetermined ratio [57]. The VLP-containing cell SUP was harvested 48 hours post-transfection, clarified by centrifugation, and equal volume from each was passaged onto fresh cell monolayers (VLP-infected cells) previously transfected with the core proteins required to support the incoming packaged minigenomes. The VLP infected cells were harvested 48 hours later and tested for optimal particle production based on incoming minigenome-encoded CAT activity. This time point was chosen because maximal VLP formation was also found to be time dependent and optimal at 48 hours post-passage.

The reproducibility of the results was verified in an independent repeat experiment. FIG. 1 shows that within the given range, and based on the levels of minigenome-encoded CAT activity, varying the concentrations of G, F and M plasmids had a bearing on VLP formation. CAT activity in the VLP infected cells appeared optimal in the boxed lanes 7 and 8 but further analysis to ensure reporter activity in the linear range indicated that the largest amount of minigenome-containing NiV VLPs were produced when the cells were transfected with the NiV M, F and G plasmid ratios of 3:1:1 as in lane 7. This ratio was used for making all the VLP preparations.

### EXAMPLE 16

### Morphologic Similarity Between NiV VLPs and the Authentic Virus

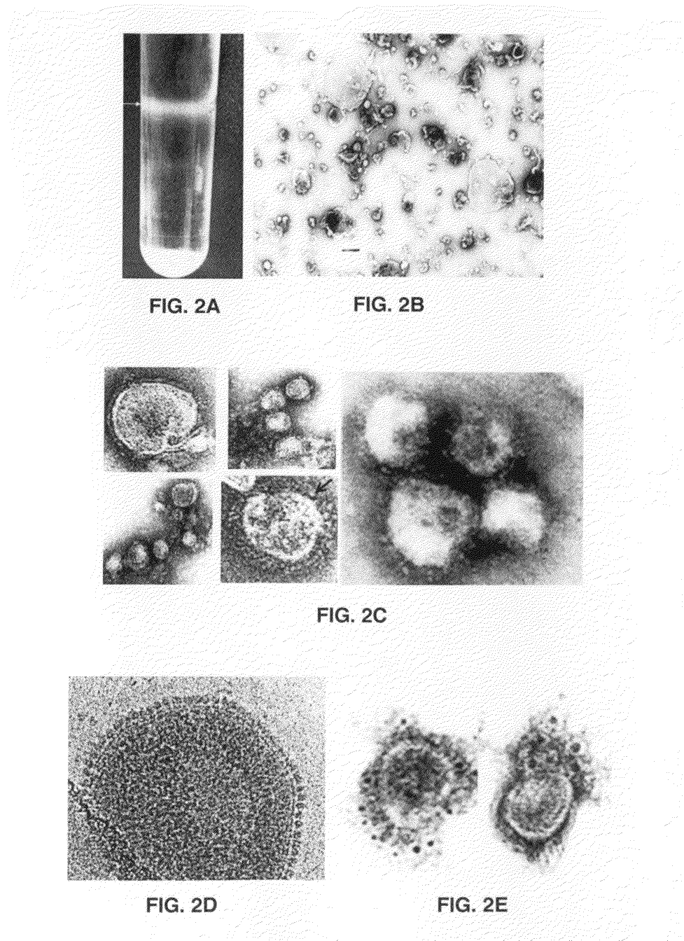

The optimized conditions were applied to transfect G, F and M expression plasmids in 293T cells grown in 10 cm dishes. The VLP-containing culture SUPs were harvested 48 hours later, and concentrated and purified as described. Briefly, the clarified SUPs were concentrated by ultracentrifugation through 20% sucrose cushion, and then purified on a discontinuous sucrose gradient. The VLP pellet was resuspended in TN buffer and viewed by EM after negative staining. FIG. 2A shows a VLP-containing band in the sucrose gradient. Viewing of the negatively stained purified particles by transmission electron microscopy (FIG. 2B) showed numerous virus-like particles. The size variation of these VLPs was consistent with the parental virus: NiV is a pleomorphic virus ranging in size from 40-1900 nm [63,64]; the sizes of the VLPs ranged from ˜40-500 nm. The particles also resembled authentic NiV morphologically, and this is seen more clearly in the magnified images presented in FIG. 2C; here, the fringe of the glycoproteins is clearly visible on the VLP surface. An occasional VLP had what appeared to be a double fringe (shown with an arrow), a feature more frequently associated with Hendra rather than NiV virus particles [64]. The image in FIG. 2D is a cryoelectron micrograph of one of the VLPs of the present invention; the overall surface appearance is virus-like, which is described as dense, ordered and repetitive [31], and it shows the surface glycoproteins and their spatial arrangement even more definitively.

### EXAMPLE 17

### Identification of NiV-Specific Proteins in the VLPs

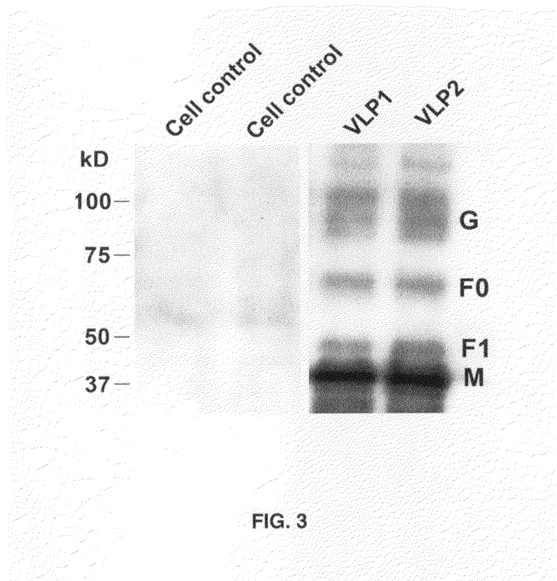

To verify whether the NiV proteins were incorporated into the VLPs as designed, purified particles were analyzed by western blotting using NiV-specific mouse antibody, and HRP-conjugated anti-mouse secondary antibody as described above. The right hand panel in the FIG. 3 shows VLP-incorporated proteins in two different preparations of NiV VLPs. The protein bands are consistent in size to NiV proteins G, F₀, F₁ and M proteins [17,48]. The relative amounts of the VLP-incorporated G and M proteins appeared to be similar to that reported in NiV virions also [17]. This was in spite of the fact that the viral proteins in that study were revealed using rabbit sera raised against bacterially expressed Hendra virus proteins. However, the ratio of the VLP-incorporated F₁ to F₀ was different from that in the virions. This difference is more likely to be a reflection of timing and protein turnover rather than the reagents used to reveal them since in a previous study, pulse chase experiments have shown that similarly to the VLP-incorporated F₁ to F₀, intracellular cleavage of the precursor NiV fusion protein by cathepsin L results in near equal mix of mature fusogenic, and the precursor forms [65]. Absence of the NiV-specific bands in two different 293T cell lysate preparations processed similarly (and shown in the left hand panel in FIG. 3) confirms specificity of the VLP-incorporated proteins.

### EXAMPLE 18

### Immunoreactivity of VLP Surface Glycoproteins

The immunoreactivity of the VLP surface glycoproteins was verified by staining purified unfixed VLPs by the immunogold labeling technique using NiV-specific mouse antiserum and 6 nm colloidal gold particle-conjugated goat anti-mouse secondary antibody (Jackson ImmunoResearch Laboratories Inc). The particles were viewed by EM after negative staining. The use of unfixed particles assured that only the surface-exposed antigens would be reactive. Numerous VLPs with the gold particles decorating their surface were seen; FIG. 2E shows two such VLPs.

### EXAMPLE 19

### Inhibition of VLP-Induced Syncytia Formation by NiV-Specific Antibodies

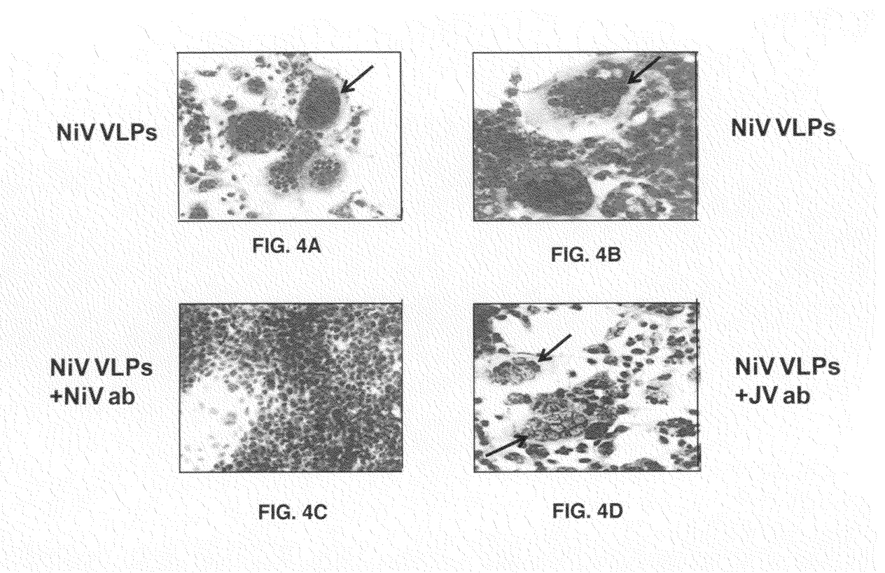

Syncytium formation is a classical feature of NiV and other paramyxovirus-induced cytopathology that can be blocked by virus-specific neutralizing antibody. A similar observation was made when 293 cells were “infected” with the NiV VLPs. Briefly, the VLPs were pre-incubated with NiV-specific antibody, Junin virus (JV)-specific antibody, and with OPTI-MEM I (Invitrogen Inc) medium only for one hour at 37° C. before inoculating onto near confluent 293 cell monolayers grown overnight in 60 mm dishes. The inoculum was removed after incubation for 3 hours at 37° C., replaced with OPTI-MEM I, and the plates were further incubated overnight at 37° C. overnight. The monolayers were then viewed for the formation of syncytia after staining with crystal violet. The results in FIG. 4 show that 293 cells exposed to NiV VLPs induced syncytium formation and that this process was neutralized by NIV-specific antibodies; prior incubation with the unrelated JV antibodies failed to block this process.

### EXAMPLE 20

### NiV VLPs as Immunogens in Balb/c Mice

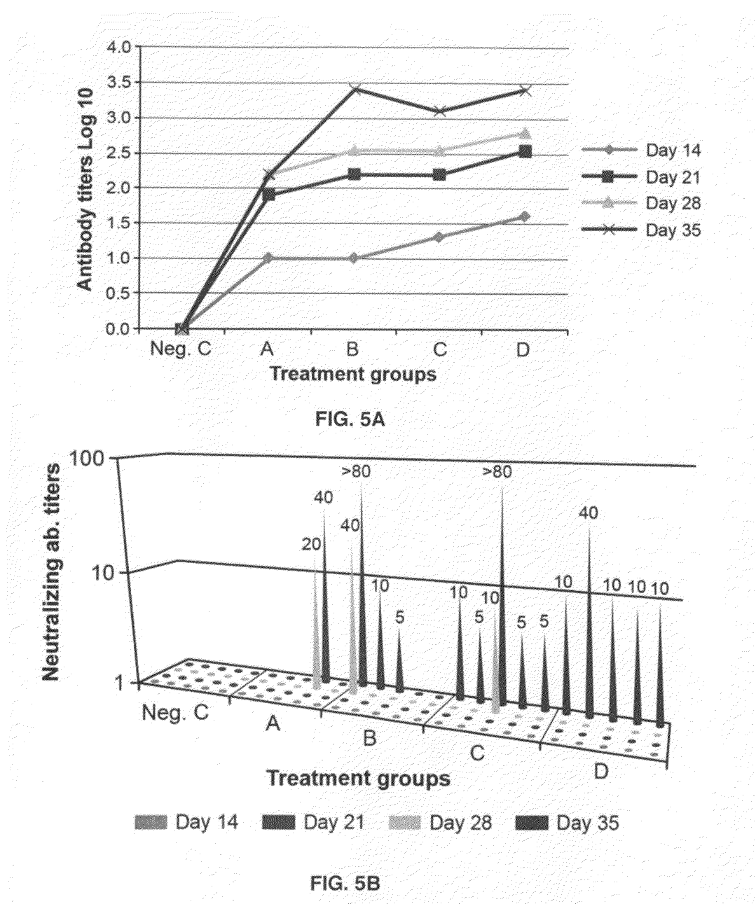

Mice in groups of five were inoculated subcutaneously with four different concentrations of purified VLPs and boosted as described above. The negative control group of five mice were inoculated with sterile endotoxin free PBS for each inoculation. The mice were bled from the submandibular vein on the day before primary inoculation, and then on days 14, 21, 28 and 35. For initial evaluation, sera from each treatment group were pooled, and the IFA method used to determine levels of NiV-specific antibodies. The results in FIG. 5A show that titers (reciprocal of the highest serum dilution showing reactivity) increased with time post primary inoculation, i.e., the highest titers (1:2560) were seen on day 35. Titers also increased with VLP dosage although by day 35, the three higher treatment groups seemed to produce similar titers. As expected, the mice in the negative control group remained nonresponsive.

All sera were tested individually by plaque reduction neutralization method by doubling dilution of each sample (1:5 to 1:80) as described above. The results (FIG. 5B) showed distinct association between VLP dosage and the ability to mount a neutralizing antibody response. Mice inoculated with the two highest VLP doses (treatment groups C and D) were each able to induce neutralizing antibodies by day 35. When samples from mice receiving the two lower concentrations of VLPs (3.5 ug/dose and 1.75 ug/dose, corresponding to treatment groups B and A respectively) were similarly tested, 3 of 5 and 1 of 5 mice respectively induced neutralizing antibody response; the titers ranged from 1:5 to >1:80. As expected, the control mice did not induce neutralizing response.

### EXAMPLE 21

### NiV VLP-Induced Activation of Genes Involved in Immune Response

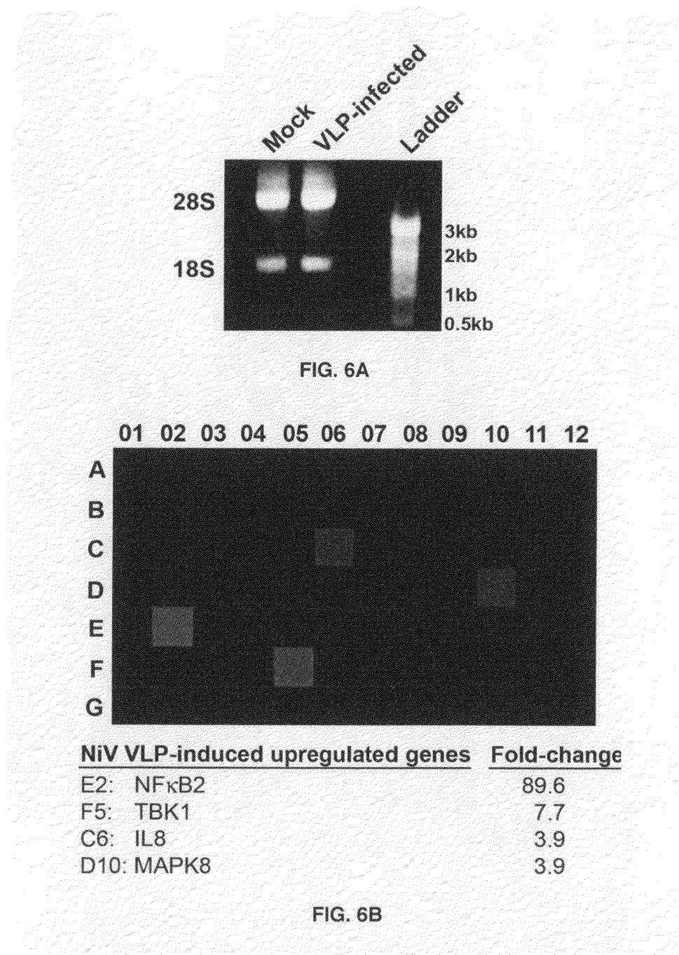

A PCR array format (SABiosciences) was used to investigate modulation in transcription profile of 84 genes involved in innate immune responses to include TLR signaling family and members of the downstream signaling pathways, NFKB, NF/IL6, IRF and JNKp38. These genes represent key sensors of non-self that signal, and ultimately shape the nature of innate immune response that modulates the type and duration of adaptive immune responses [66,67]. The differential expression of genes in VLP-exposed 293 cells relative to the “mock” infected 293 cells was measured by real-time PCR. Same concentration of total cell RNA from the VLP-stimulated and the “mock” stimulated control cells were used for first strand synthesis and Sybr green PCR amplification of the relevant genes as described above. The integrity of RNA in each sample was confirmed by gel electrophoresis (FIG. 6A). Data representing the differential transcription profile of VLP exposed vs. “mock” stimulated cells is shown as a heat map (FIG. 6B). A 4-fold cutoff threshold was used to determine modulation in gene expression. There was significant VLP-stimulated up-regulation (89 fold and 7 fold) in the expression of NFKB2 and TBK1 genes respectively. Close to four fold (3.9 fold) up-regulation was noted also in IL-8 and MAPK8 genes. NFKB2 and IL-8 are target genes in the downstream NFKB pathway, and TBK1 which are in the IRF and JNK/p38 pathways respectively.

### Discussion

Using a minigenome-based functional assay, the present invention established conditions (described above and shown in FIG. 1) that allowed production of substantial quantities of NiV VLPs. These particles are functionally assembled, biologically active and are able to induce innate immune responses, and a neutralizing antibody response. Native VLPs have been used to study various aspects of the virus lifecycle, as carriers to deliver heterologous proteins for vaccination, and to deliver small molecules for gene therapy purposes. Importantly, they have been used highly effectively as vaccines in their native form [31,32,39,40,41].

No safe and protective vaccine for NiV disease has been developed for humans. The two vaccination strategies that have been explored are the canary pox-based vector approach [26] and soluble subunit approach [28,29]. NiV vaccine by the former method is undergoing development as a veterinary vaccine [26]. The same approach is being evaluated for human use vaccines, mainly for the prevention of HIV and AIDS [27]. The subunit approach has limitations as mentioned above [28,29,30,31,32]. One particular challenge revealed by studies that tested a soluble NiV G protein-based subunit vaccine formulated with adjuvant is the potential difficulty of eradicating infection in the central nervous system. In that study [28], live virus was present in the brain of one cat, and viral RNA was present throughout the 21 day post-challenge period in the brains of the remaining challenged animals. A recently reported vaccination strategy [34] requires simultaneous inoculation of two VSVΔG vectors, one expressing NiV G, and the other expressing NiV F proteins. It was of interest to note that supernatants of cells co-infected with these two defective viruses were infectious and could be passaged indefinitely in the absence of VSV G trans-complementation. This vaccination approach seems promising since self-propagated stock of these two viruses induced robust neutralizing antibody response in mice. However, potential pathogenicity of VSV-based vaccine vectors remains a concern [34,35]. The potential of a recombination event resulting in a single VSV vector virus expressing both these NiV proteins is unlikely, but it may still be problematic for a human use vaccine.

Native VLPs like the ones of the present invention allow the viral proteins to be presented to the immune system in the same conformation as in the virion for effective B and T cell response [31]. VLPs are particularly effective in producing a protective antibody response because of their virus-like size range, their particulate nature, and their virus-like dense, repetitive and ordered surface structure [31,36]. The spacing of the antigenic epitopes on the VLP is also optimal for B cell activation [31]: EM analysis showed that the particles of the present invention resembled the real virus in terms of size and surface structure [63,64]. The image in FIG. 2D is the first elucidation of VLP structure of any paramyxovirus imaged by CryoEM, and it provides a careful assessment of their morphology; it alludes to a surface similar to that revealed for measles virus by the same imaging technique (Dr. Elizabeth Wright, Emory University). The proteins on the VLP surface are clearly visible here; the average distance between the spikes was 9.13 nm and standard deviation was 1.72 nm. This is of interest given that epitopes spaced between 5 and 10 nm are known to be sufficient to drive optimal B cell activation [36].

NiV M, F, G and N protein-containing VLPs consistent in size and morphology to the parental virus have also been reported in a study which evaluated protein-protein interaction that facilitate VLP formation [48]. However, in that study, most of the particle-incorporated NiV F protein was predominantly in the uncleaved precursor form. This finding is clearly distinct since the VLPs of the present invention contained substantial amounts of cleaved F protein, and this may have been related to ratios of the interacting proteins expressed in 293T transfected cells. In a recent study of NDV VLPs [49], the particle-incorporated proteins were reported to have virus-like protein ratios, but the F protein remained in its precursor form because the cleavage site required to produce the fusion competent form was mutated by design. What effect a VLP-incorporated non-fusogenic F protein may have, relative to the fusogenic form, on the level and quality of VLP-induced immune response is not clear at present since, difference in immunogenicity between fusion-competent and fusion-defective VLPs has not been experimentally evaluated so far. However, a recent report suggests that viral fusogenic membrane glycoproteins may enhance vaccine potency [68].

Immunogold labeling of the unfixed NiV VLPs confirmed that the surface proteins in the VLPs of the present invention were functionally assembled and they were biologically active (FIG. 2E). The presence of biologically active G and F proteins on the VLP surface deduced by the fact that they were able to induce the formation of syncytia in 293 cells (FIG. 4); this is a process that requires the interaction of both the surface glycoproteins, the attachment protein G, and the fusion competent F protein, when they come in contact with the cognate receptor-bearing cells. (The attachment protein in different paramyxoviruses is referred as G, HN, or H, depending on the virus. For example in Nipah virus and respiratory syncytial virus it is G protein, but for measles virus it is H protein). Formation of syncytia or multinucleated cells in replication competent enveloped viruses, especially paramyxoviruses, is induced by a process that is described as “fusion from within”, and it can be blocked or neutralized by prior treatment of the virus with specific antisera. In contrast, “fusion from without” is induced by non-replicating viruses at high multiplicities of infection, and it too can be blocked by pretreatment with virus-specific antibodies ([69], [70]). The non-replicating particles of the present invention likewise induced syncytia formation in 293 cells that could be neutralized with NiV-specific antibodies (FIG. 4). This is the first study describing fusion from without induced by VLPs of any paramyxovirus, or any other enveloped viruses, although it has been described for the VLPs of the non-enveloped rotavirus [71]. The mechanism(s) of fusion from without is not clear but two models have been proposed [72,73]. One model proposes that particles connecting adjacent cells effectively promote fusion between them, and the other is that when particles decorated with the surface glycoproteins fuse with the target cell membrane, the glycoprotein complexes diffuse freely in the lipid bilayer, and mimic fusion from within.

Neutralizing antibody response is the critical correlate of protection mediated by prophylactic vaccines [53,54] and native VLPs promise to be highly effective prophylactic vaccines for paramyxoviruses like NiV, and others like NDV and measles where neutralizing immune response is known to play a pivotal role in protection against disease [53,54,55,74]. The VLPs of the present invention were highly effective immunogens, and all, especially in the three higher treatment groups produced high levels of response by day 35 (FIG. 5A). Importantly, NiV VLPs were able to induce neutralizing antibodies. This response was clearly dose-dependent (FIG. 5B). All ten mice receiving a primary inoculation of 7 or 14 μg VLPs (subgroup C and D) were able to produce such response; but even of those animals that received a first dose of only 3.5 or 1.75 mg/mouse (treatment group B and A respectively), 3 of 5 and 1 of 5 produced neutralizing antibodies. Neutralization antibody response was first seen on day 28, and increasing titers were seen in some animals within a week of it; this response, induced by these non-replicating and potentially safe particles, formulated without adjuvant, compares favorably with the levels of such response induced at an equivalent time point by some replication competent pseudotype viruses [34].

Immunogenicity to native VLPs has been reported previously for one other paramyxovirus namely NDV [49]. In that report, immune response to NDV VLPs was evaluated by primary inoculation of mice intraperitoneally with VLP concentrations ranging between 10 and 40 μg, and a booster dose of 10 μg, without adjuvant. NDV-specific titers by ELISA were high in each mouse in each treatment group. Neutralizing antibody response to 20 and 40 μg of these particles was also detected.

The nature of innate immune response dictates the type and duration of adaptive immune response [67,75]. The mechanism by which NiV VLPs are recognized by host cells and trigger the induction of innate immune response, and how this translates into effective adaptive immunity is not known. With the experimental conditions as described, VLP-induced activation of certain genes known to be involved in the induction of an effective innate immune response [75]. Results presented in the heat map in FIG. 6 show that relative to the “mock” treated cells, NFKB2 gene (in the NFKB pathway) was up-regulated 89 fold as a result of VLP exposure, and TBK1 (in the IRF pathway) was 7 fold higher. In the light of these findings, PCR array expression profiles of the same set of 84 genes are examined in 293 and other cells at earlier and later time points to identify their upstream effectors, and NiV VLP-responsive signaling networks. In this respect, the murine system, with the many available immunological reagents and knockout strains may provide the best system to identify these host sensors. Currently there is minimal information on live NiV infection-responsive cell-signaling changes [76] and there is none on array-based transcriptional alterations for comparative analysis. Likewise, it has not been possible to compare the NiV VLP-induced transcription modulation with those induced by other paramyxovirus VLPs. A growing number of reports point to viral surface glycolproteins as relevant in host cell signaling and triggering of innate immune response. Particles like NiV VLPs, with many virus-like properties (including their surface glycoproteins organized to resemble the parental virus, FIG. 2D) would induce an effective innate immune response for the promotion of the desired adaptive immunity [67,76].

Finally, as described above, the VLPs of the present invention were highly effective as immunogens, able to induce neutralizing antibody response in all animals with primary inoculation of as little as 7 μg VLP protein each. Fusogenic property of these VLPs may be critically relevant.

In conclusion, substantial quantities of NiV VLPs needed to characterize NiV VLPs were produced, their many virus-like properties and their effectiveness as immunogens were demonstrated.

The following references were cited herein:
1. Chan et al. (2002) Epidemiol Infect 128: 93-98.
2. Chua et al. (1999) Lancet 354: 1257-1259.
3. Chua et al. (2000) Science 288: 1432-1435.
4. Chua K B (2003) Nipah virus outbreak in Malaysia. J Clin Virol 26: 265-275.
5. Chadha et al. (2006) Emerg Infect Dis 12: 235-240.
6. Hsu et al. (2004) Emerg Infect Dis 10: 2082-2087.
7. ICDDR BCfhapr (2004) Health and Science Bulletin 2: 7-11.
8. WHO (2004) The Weekly Epidemiological Record (WER) 79: 168-171.
9. ICDDR BCfhapr (2005) Health and Science Bulletin 3: 1-5.
10. Gurley, et al. (2007) Emerg Infect Dis 13: 1031-1037.
11. Gurley et al. (2007) Infect Control Hosp Epidemiol 28: 740-742.
12. ICDDR BCfhapr (2004) Health and Science Bulletin 2: 5-9.
13. Lam S K (2003) Antiviral Res 57: 113-119.
14. Eaton et al., (2006) Nat Rev Microbiol 4: 23-35.
15. Harcourt et al. (2000) Virology 271: 334-349.
16. Harcourt et al. (2005) 2004. Emerg Infect Dis 11: 1594-1597.
17. Wang et al. (2001) Microbes Infect 3: 279-287.
18. Wong et al. (2002) Am J Pathol 161: 2153-2167.
19. Bossart et al. (2007) J Virol Methods 142: 29-40.
20. Ciancanelli M J, Basler C F (2006) J Virol 80: 12070-12078.
21. Bonaparte et al. (2005) Proc Natl Acad Sci USA 102: 10652-10657.
22. Negrete et al. (2005) Nature 436: 401-405.
23. Negrete et al. (2006) PLoS Pathog 2: e7.
24. Schmitt A P, Lamb R A (2004) Curr Top Microbiol Immunol 283: 145-196.
25. Takimoto T, Portner A (2004) Virus Res 106: 133-145.
26. Weingartl et al. (2006) J Virol 80: 7929-7938.
27. Pantaleo et al., (2010) Curr Opin HIV AIDS 5: 391-396.
28. McEachern et al. (2008) Vaccine 26: 3842-3852.
29. Mungall et al. (2006) J Virol 80: 12293-12302.
30. Bachmann et al. (1993) Science 262: 1448-1451.
31. Jennings G T, Bachmann M F (2007) Curr Mol Med 7: 143-155.
32. Noad R, Roy P (2003) Trends Microbiol 11: 438-444.
33. Defang et al., (2010) Vaccine 29: 212-220.
34. Chattopadhyay A, Rose J K (2010) J Virol.
35. Johnson et al. (2007) Virology 360: 36-49.
36. Jegerlehner et al. (2002) Vaccine 20: 3104-3112.
37. Akahata et al. (2010) Nat Med 16: 334-338.
38. Chackerian B (2007) Expert Rev Vaccines 6: 381-390.
39. Grgacic E V, Anderson D A (2006) Methods 40: 60-65.
40. Jennings G T, Bachmann M F (2008) Biol Chem 389: 521-536.
41. Jennings et al., (2009) Annu Rev Pharmacol Toxicol 49: 303-326.
42. Warfield, et al. (2007) J Infect Dis 196 Suppl 2: S430-437.
43. Coronel E C, Mutt K G, Takimoto T, Portner A (1999) J Virol 73: 7035-7038.
44. Pantua et al., (2006) J Virol 80: 11062-11073.
45. Schmitt A P, Leser G P, Waning D L, Lamb R A (2002) J Virol 76: 3952-3964.
46. Sugahara et al. (2004) Virology 325: 1-10.
47. Teng M N, Collins P L (1998) J Virol 72: 5707-5716.
48. Patch et al., (2007) Virol J 4: 1.
49. McGinnes et al. (2010) J Virol 84: 4513-4523.
50. Bossart et al. (2005) J Virol 79: 6690-6702.
51. Bossart et al. (2002) J Virol 76: 11186-11198.
52. Lamb R A, and Kolakofsky, D (2001) Paramyxoviridae: the viruses and their replication. In: D. M. Knipe et al., eds. Fields virology. 4th ed. Philadelphia, Pa.: Lippincott, Williams & Wilkins. pp. 1305-1340.
53. Plotkin S A (2010) Clin Vaccine Immunol 17: 1055-1065.
54. Reading S A, Dimmock N J (2007) Arch Virol 152: 1047-1059.
55. Guillaume et al. (2006) J Virol 80: 1972-1978.
56. Niwa H, Yamamura K, Miyazaki J (1991) Gene 108: 193-199.
57. Walpita P, Peters C J (2007) J Gen Virol 88: 2542-2551.
58. Lokugamage et al. (2008) Vaccine 26: 797-808.
59. Pattnaik A K, Wertz G W (1991) Proc Natl Acad Sci USA 88: 1379-1383.
60. Whelan S P, Wertz G W (1999) J Virol 73: 307-315.
61. Sherman M B, Weaver S C (2010) J Virol 84: 9775-9782.
62. Walpita P, Flick R (2005) FEMS Microbiol Lett 244: 9-18.
63. Goldsmith, et al. (2003) Virus Res 92: 89-98.
64. Hyatt et al., (2001) Microbes Infect 3: 297-306.
65. Pager (2006) Virology 346: 251-257.
66. Beutler B A (2009) TLRs and innate immunity. Blood 113: 1399-1407.
67. Iwasaki A, Medzhitov R (2010) Science 327: 291-295.
68. Mao et al. (2010) J Virol 84: 2331-2339.
69. Bratt M A, Gallaher W R (1969) Proc Natl Acad Sci USA 64: 536-543.
70. Collins S E, Noyce R S, Mossman K L (2004) J Virol 78: 1706-1717.
71. Gilbert J M, Greenberg H B (1997) J Virol 71: 4555-4563.
72. Clavel F, Charneau P (1994) J Virol 68: 1179-1185.
73. Knutton S, Jackson D, Ford M (1977) J Cell Sci 28: 179-188.
74. Guillaume et al. (2004) J Virol 78: 834-840.
75. Iwasaki A, Medzhitov R (2004) Nat Immunol 5: 987-995.
76. Lo et al. (2010) Virology 404: 78-88.
77. Wong K T (2003) Am J pathol 5: 2127-2137.

Any patents or publications mentioned in this specification are indicative of the levels of those skilled in the art to which the invention pertains. These patents and publications are herein incorporated by reference to the same extent as if each individual publication was specifically and individually incorporated by reference.

One skilled in the art will readily appreciate that the present invention is well adapted to carry out the objects and obtain the ends and advantages mentioned, as well as those inherent therein. It will be apparent to those skilled in the art that various modifications and variations can be made in practicing the present invention without departing from the spirit or scope of the invention.

What is claimed is:

1. Paramyxovirus virus-like particles, wherein said virus-like particles are composed of:
surface glycoprotein G;
surface glycoprotein F; and
matrix protein M.

2. The paramyxovirus virus-like particles of claim 1, wherein said paramyxovirus is selected from the group consisting of Avulavirus, Henipavirus, Morbillivirus, Respirovirus, Rubulavirus, TPMV-like viruses, Pneumovirinae, Pneumovirus and Metapneumovirus.

3. The paramyxovirus virus-like particles of claim 1, wherein said Henipavirus is Nipah Virus.

4. The paramyxovirus virus-like particles of claim 1, wherein said G and F proteins mediate attachment and entry into the host cell and said virus-like particles activate innate immune signaling in cells infected with paramyxovirus Virus.

5. The paramyxovirus virus-like particles of claim 1, wherein said virus-like particles induce robust neutralizing antibody response.

6. The paramyxovirus virus-like particles of claim 1, wherein said virus-like particles are fusogenic and induce syncytia formation.

7. The paramyxovirus virus-like particles of claim 1, wherein said virus-like particles have biologically active G and F proteins on the particle surface.

8. The paramyxovirus virus-like particles of claim 1, wherein said virus-like particles are purified.

9. The paramyxovirus virus-like particles of claim 1, further comprising a protein selected from the group consisting of an N protein, an L protein and an P protein of the virus.

10. The paramyxovirus virus-like particles of claim 1, further comprising a protein selected from the group consisting of a C protein and a W protein of the virus.

11. The paramyxovirus virus-like particles of claim 1, further comprising encapsidating synthetic RNAs.

12. The paramyxovirus virus-like particles of claim 1, wherein G in the range of 0.7 to 1.3, F in the range of 0.7 to 1.3 and M in the range of 2.6 to 3.4.

13. The paramyxovirus virus-like particles of claim 1, wherein G in the range of 0.8 to 1.2, F in the range of 0.8 to 1.2 and M in the range of 2.7 to 3.3.

14. The paramyxovirus virus-like particles of claim 1, wherein G in the range of 0.9 to 1.1, F in the range of 0.9 to 1.1 and M in the range of 2.8 to 3.2.

15. The paramyxovirus virus-like particles of claim 1, wherein G in the range of 0.9 to 1.1, F in the range of 0.9 to 1.1 and M in the range of 2.9 to 3.1.

16. An expression vector comprising a nucleotide sequence that encodes the virus-like particles of claim 1.

17. A vaccine comprising the virus-like particles of claim 1 and a pharmaceutically acceptable carrier or an adjuvant.

18. A method of vaccinating a subject against paramyxovirus infection comprising administering the vaccine of claim 17.

19. The method of claim 18, wherein said subject is a human.

20. The method of claim 18, wherein said administering is intramuscular or subcutaneous.

21. The method of claim 18, wherein said administering is in a single dose.

22. The method of claim 18, wherein said administering comprises about 5 micrograms to about 200 micrograms of said virus-like particles.
